(19) United States
(12) Patent Application Publication
(10) Pub. No.: US 2013/0110043 A1
(43) Pub. Date: May 2, 2013

(54) Microneedle Intradermal Drug Delivery Device with Auto-Disable Functionality

(71) Applicant: Yotam Levin, Nes Ziona (IL)

(72) Inventor: Yotam Levin, Nes Ziona (IL)

(73) Assignee: NanoPass Technologies Ltd., Nes Ziona, IL (US)

(21) Appl. No.: 13/661,059

(22) Filed: Oct. 26, 2012

Related U.S. Application Data
(60) Provisional application No. 61/551,448, filed on Oct. 26, 2011.

(51) Int. Cl. A61M 5/50

(52) U.S. Cl. 604/110

(57) ABSTRACT

A microneedle intradermal drug delivery device with auto-disable functionality includes a syringe (12) having a plunger (14) displaceable along a barrel (16) and a microneedle adapter (20) with hollow microneedle(s) (22). The microneedle adapter (20) and the syringe (12) are configured for irreversible engagement such that, after attachment of the microneedle adapter (20) to the syringe (12), the microneedle adapter (20) is resistant to non-destructive manual removal from the syringe (12). Also provided are a self-locking plunger configuration (54, 56, 58) and a sliding safety shield (62).

### FIELD AND BACKGROUND OF THE INVENTION

The present invention relates to systems and methods providing auto disable (AD) features to a microneedle device and, in particular, systems and methods for performing such using microneedle-syringe mating arrangements. The invention also provides various other auto-disable or safety features not necessarily limited to microneedle applications.

Microneedles, defined herein as sharp projections with a total exposed length of no more than 1 millimeter, may be used for intradermal (ID) injections of fluids. Such injections may facilitate dose sparing. For example it has been previously demonstrated that reduced doses of a vaccine delivered intradermally can produce equivalent immune responses (or immunogenicity) with the full dose (and volume) of intra-muscular (IM) injection (Van Damme P, et al. Safety and efficacy of a novel microneedle device for dose sparing intradermal influenza vaccination in healthy adults. Vaccine (2008), oi:10.1016/j.vaccine.2008.10.077), as well as sometimes improve the immune response despite the use of lesser doses (Holland D, Booy R, De Looze F, Eizenberg P, McDonald J, Karrasch J, et al. Intradermal influenza vaccine administered using a new microinjection system produces superior immunogenicity in elderly adults: a randomized controlled trial. J Infect Dis 2008; 198:6508 and Hung I F N, Levin Y, To K W W, Chan K H, Zhang A J, Li P, Li C, Xu T, Wong T Y and Yuen K Y. Dose sparing intradermal trivalent influenza (2010/2011) vaccination overcomes reduced immunogenicity of the 2009 H1N1 strain. Vaccine. In Press. Available online 17 Aug. 2012. http://dx.doi.org/10.1016/j.vaccine.2012.08.014).

For some immunization (as well as therapeutic or diagnostic) applications, and for some markets (e.g. the developing countries markets, or globally for drug users) there is a need to “auto disable” the device, i.e., to prevent reuse of the drug delivery device, which might lead to cross infection and contamination.

Implementation of an auto-disable syringe with a microneedle injection interface presents particular challenges. Specifically, the short length of the microneedles prevents the injection interface from being used to withdraw a drug from a storage vial. Instead, a dedicated filling adapter (either a filling needle or a vial adapter) must first be used, and a microneedle adapter is then substituted for the filling adapter. The interchangeability of the adapters tends to facilitate improper repeat usage.

There is therefore a need for a microneedle intradermal drug delivery device which would provide auto-disable functionality.

### SUMMARY OF THE INVENTION

An aspect of the present invention is a microneedle intradermal drug delivery device providing auto-disable functionality.

According to the teachings of an embodiment of the present invention there is provided, an intradermal drug delivery device comprising: (a) a syringe having a plunger displaceable along a barrel for drawing a quantity of a liquid drug through an outlet and expelling the liquid drug through the outlet; and (b) a microneedle adapter including at least one hollow microneedle, the microneedle adapter being configured to mate with the syringe so as to provide a leak-free flow path from the outlet through the at least one hollow microneedle for delivering the liquid drug intradermally, wherein the microneedle adapter and the syringe are configured for irreversible engagement such that, after attachment of the microneedle adapter to the syringe, the microneedle adapter is resistant to non-destructive manual removal from the syringe.

According to a further feature of an embodiment of the present invention, the outlet runs through a male conical fitting having a conical angle of less than 5.5%, and wherein the microneedle adapter is formed with a female conical fitting configured to mate with the male conical fitting.

According to a further feature of an embodiment of the present invention, the outlet runs through a male conical fitting formed with a circumferential groove, and wherein the microneedle adapter is formed with a female conical fitting having at least one ridge, the female conical fitting being configured to mate with the male conical fitting with the at least one ridge engaging the groove.

According to a further feature of an embodiment of the present invention, the outlet runs through a male conical fitting fowled with at least one projecting ridge, and wherein the microneedle adapter is formed with a female conical fitting having a circumferential groove, the female conical fitting being configured to mate with the male conical fitting with the at least one ridge engaging the groove.

According to a further feature of an embodiment of the present invention, the syringe is formed with at least one resilient engagement portion deployed to provide snap-engagement with a corresponding feature of the microneedle adapter.

According to a further feature of an embodiment of the present invention, the microneedle adapter is formed with at least one resilient engagement portion deployed to provide snap-engagement with a corresponding feature of the syringe.

According to a further feature of an embodiment of the present invention, the at least one hollow microneedle is integrally formed with a substrate from a single crystal material.

According to a further feature of an embodiment of the present invention, the at least one hollow microneedle is formed with at least one upright surface, an inclined surface intersecting with the at least one upright surface, and a fluid flow bore extending through the substrate and intersecting with the inclined surface.

According to a further feature of an embodiment of the present invention, there is also provided a vial adapter configured for releasable engagement with the outlet for filling of the syringe.

According to a further feature of an embodiment of the present invention, the plunger is formed with a plunger extension extending from a seal of the plunger and configured to advance within the outlet as the plunger is advanced, thereby reducing a dead-space of the syringe.

According to a further feature of an embodiment of the present invention, the plunger extension further comprises a resilient tip configured to extend beyond the outlet in a fully advanced position of the plunger, the resilient tip being configured to expand laterally so as to engage a region of the syringe around the outlet, thereby inhibiting withdrawal of the plunger extension.

According to a further feature of an embodiment of the present invention, the plunger further comprises a reduced-strength region configured to break under traction applied to withdraw the plunger after engagement of the resilient tip.

According to a further feature of an embodiment of the present invention, the syringe is an auto-disable syringe preventing refilling.

According to a further feature of an embodiment of the present invention, there is also provided a sheath circumscribing the barrel, the sheath being selectively displaceable to an advanced position in which the sheath covers the microneedle adapter, the sheath and the syringe having interlocking features configured to prevent retraction of the sheath from the advanced position.

According to a further feature of an embodiment of the present invention, the at least one hollow microneedle is implemented as a plurality of microneedles.

According to a further feature of an embodiment of the present invention, the at least one hollow microneedle is implemented as a linear array of at least three microneedles.

There is also provided according to an embodiment of the present invention, an auto-disable syringe comprising: (a) a syringe body comprising a barrel and terminating at an outlet; and (b) a plunger having a shaft for driving a seal along the barrel so as to deliver a quantity of liquid through the outlet, wherein the plunger is formed with a plunger extension extending from the seal of the plunger and configured to advance within the outlet as the plunger is advanced, and wherein the plunger extension further comprises a resilient tip configured to extend beyond the outlet in a fully advanced position of the plunger, the resilient tip being configured to expand laterally so as to engage a region of the syringe around the outlet, thereby inhibiting withdrawal of the plunger extension.

According to a further feature of an embodiment of the present invention, the shaft, the plunger extension and the resilient tip are integrally formed as a single element.

According to a further feature of an embodiment of the present invention, the plunger further comprises a reduced-strength region configured to break when force is applied to withdraw the plunger after engagement of the resilient tip.

### BRIEF DESCRIPTION OF THE DRAWINGS

The invention is herein described, by way of example only, with reference to the accompanying drawings, wherein:

FIG. 1 is a schematic cross-sectional view of a drug delivery device employing a microneedle adapter and a syringe, constructed and operative according to certain embodiments of the present invention, but without showing details of an irreversible engagement arrangement between the microneedle adapter and the syringe;

FIG. 2 is an enlarged view showing a first implementation of an irreversible engagement arrangement for use in the drug delivery device of FIG. 1;

FIG. 3 is an enlarged view showing a second implementation of an irreversible engagement arrangement for use in the drug delivery device of FIG. 1;

FIG. 4 is an enlarged view showing a third implementation of an irreversible engagement arrangement for use in the drug delivery device of FIG. 1;

FIG. 5 is an enlarged view showing a fourth implementation of an irreversible engagement arrangement for use in the drug delivery device of FIG. 1;

FIG. 6 is an enlarged view showing a fifth implementation of an irreversible engagement arrangement for use in the drug delivery device of FIG. 1;

FIG. 7 is a schematic side view of a vial adapter for use with the syringe from the drug delivery device of FIG. 1;

FIG. 8A is a partial cross-sectional view taken through a drug delivery device according to a further aspect of the present invention including a self-locking plunger configuration;

FIG. 8B is an isometric view of a distal part of the plunger configuration from the device of FIG. 8A;

FIGS. 9A and 9B are isometric views of a drug delivery device according to a further aspect of the present invention including a safety cover for rendering the microneedle adapter inaccessible after use, the cover being shown in its normal retracted position and in its deployed safety position, respectively;

FIGS. 10A and 10B are cross-sectional views taken through the device of FIGS. 9A and 9B in the respective positions of FIGS. 9A and 913; and

FIGS. 11A and 11B are views similar to FIGS. 10A and 10B, respectively, illustrating the safety cover feature implemented together with the self-locking plunger configuration of FIGS. 8A and 8B.

### DESCRIPTION OF THE PREFERRED EMBODIMENTS

An aspect of the present invention is a microneedle intradermal drug delivery device providing auto-disable functionality.

The principles and operation of drug delivery devices according to the present invention may be better understood with reference to the drawings and the accompanying description.

By way of introduction, an aspect of the present invention takes advantage of the inherent difficulty of refilling a drug delivery device via a microneedle adapter to provide auto-disable functionality. Specifically, according to certain preferred implementations of the present invention, by rendering attachment of a microneedle adapter to a syringe irreversible, this inherently limits the user's ability to refill the device for repeat usage.

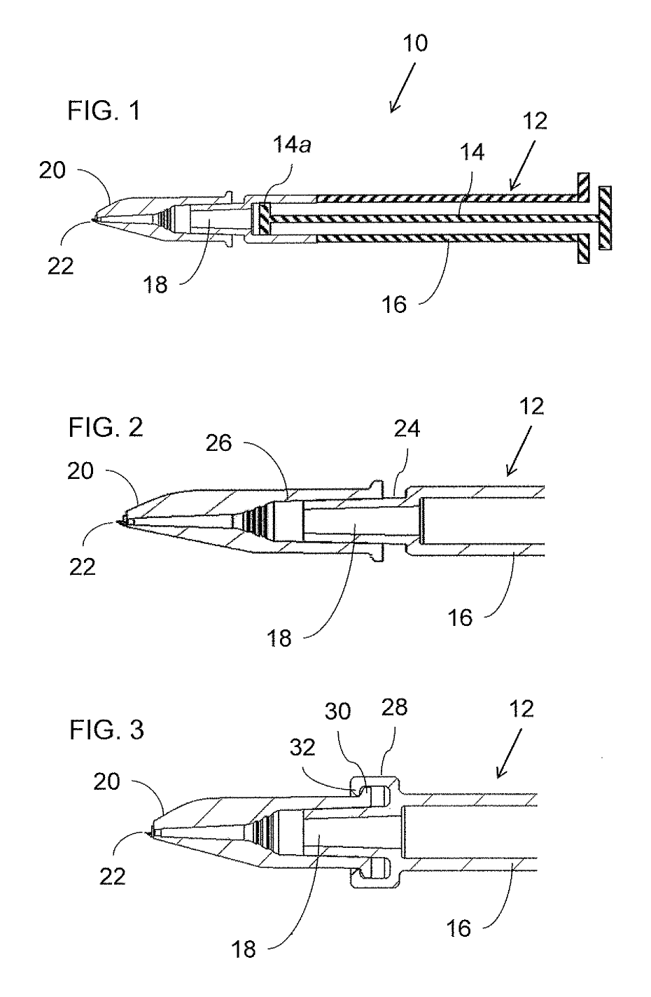

Referring now to the drawings, FIG. 1 shows a generic overview of an intradermal drug delivery device, generally designated 10, according to an aspect of the present invention. Generally speaking, drug delivery device 10 includes a syringe 12 having a plunger 14 displaceable along a barrel 16 for drawing a quantity of a liquid drug through an outlet 18 and expelling the liquid drug through the outlet. A microneedle adapter 20, including at least one hollow microneedle 22, is configured to mate with the syringe so as to provide a leak-free flow path from outlet 18 through the at least one hollow microneedle 22 for delivering the liquid drug intradermally.

It is a particular feature of this aspect of the present invention that microneedle adapter 20 and syringe 12 are configured for irreversible engagement such that, after attachment of microneedle adapter 20 to syringe 12, microneedle adapter 20 is resistant to non-destructive manual removal from syringe 12. Since the microneedles are too short to penetrate the septum of a drug vial, irreversible engagement of the microneedle adapter with the syringe inherently prevents refilling of the syringe from a drug vial.

FIGS. 2-6 illustrate a number of non-limiting but particularly preferred implementations for the irreversible engagement of FIG. 1. Referring first to FIG. 2, this illustrates an implementation in which outlet 18 runs through a male conical fitting 24 having a conical angle of less than 5.5%, and wherein the microneedle adapter is formed with a female conical fitting 26 configured to mate with the male conical fitting. Fittings 24 and 26 can be regarded as modified Luer connectors. Standard Luer connectors are formed with a conical angle of 6% which is chosen to provide releasable retention of the fittings during use. As the conical angle is reduced (i.e., becomes less steeply tapered), the resulting clamping force between the two components becomes much greater. By selection of a suitable conical angle together with suitable choice of materials, it is possible to achieve a push-on connection which cannot readily be separated by hand.

Turning now to FIG. 3, this shows a further option in which syringe 12 is formed with at least one resilient engagement portion 28 deployed to provide snap-engagement with a corresponding feature 30 of microneedle adapter 20. In the preferred implementation illustrated here, the feature 30 is a flange at the rear (proximal) end of microneedle adapter 20, and resilient engagement portion 28 is a peripheral collar extending around the flange and terminating at an inwardly projecting ridge or set of teeth 32. The collar is typically slotted to provide the desired degree of flexibility. The collar may be replaced by a number of separate clasps spaced around the periphery of flange 30, but in certain cases, a continuous or near-continuous collar is preferred for providing enhanced tamper resistance. The ridge or set of teeth are preferably directional, with an inclined distal surface to facilitate insertion of the flange and a radial or even undercut rear-facing surface for secure engagement of the flange.

The sealed interconnection between the syringe 12 and microneedle adapter 20 is preferably provided (here and in all other embodiments) by male/female Luer connector surfaces, which may be standard taper surfaces or the modified taper angle surfaces described with reference to FIG. 2 above.

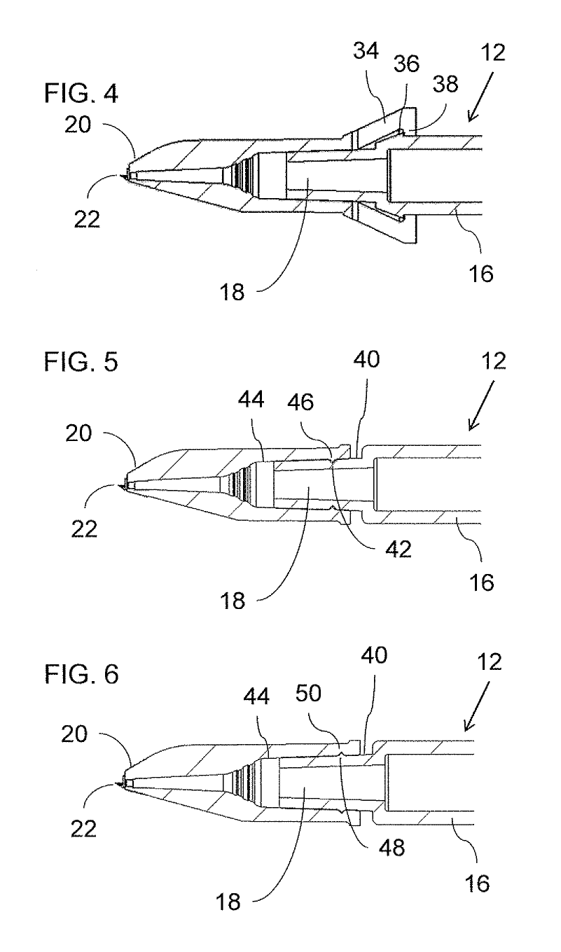

FIG. 4 shows an alternative preferred implementation in which microneedle adapter 20 is formed with at least one resilient engagement portion 34 deployed to provide snap-engagement with a corresponding feature 36 of syringe 12. In the preferred implementation illustrated here, feature 36 is a circumferential ridge extending around barrel 16 near its distal end, and engagement portions 34 are resilient arms terminating in an inward projection 38 which engages behind ridge 36.

Turning now to FIGS. 5 and 6, these show further implementations in which irreversible interconnection is achieved by modification of the conical (Luer) fixtures themselves. In the case of FIG. 5, outlet 18 runs through a male conical fitting 40 formed with a circumferential groove 42, and microneedle adapter 20 is formed with a female conical fitting 44 having at least one ridge 46. Female conical fitting 44 is configured to mate with male conical fitting 40 with ridge(s) 46 engaging groove 42. The groove and ridge may have a directional “barbed” form to facilitate engagement and resist disengagement. Optionally, the broader part of the conical fitting may be formed with slots to provide extra flexibility during the engagement process, so long as sufficient non-slotted overlap between the male and female fittings remains to ensure a full seal between them.

FIG. 6 shows an inverted implementation in which male conical fitting 40 is formed with at least one projecting ridge 48 and female conical fitting 44 is fowled with a circumferential groove 50. In all other respects, the implementation of FIG. 6 is analogous in structure and function to that of FIG. 5.

In all of the above cases, microneedle adapter 20 has been described as having at least one hollow microneedle 22. Most preferably, the microneedle is integrally formed with a substrate from a single crystal material, typically silicon. A particularly preferred choice of microneedle structure is the hollow micropyramid commercially available from Nanopass Technologies Ltd. under the trade name MICRONJET 600. These microneedles are formed with at least one surface which is upright relative to the plane of the substrate surface and an inclined surface intersecting with the upright surface(s) to form an asymmetric pyramid structure. A fluid flow bore extends through the substrate and intersects with the inclined surface. Additional details about the manufacturing process for such needles may be found in U.S. Pat. No. 7,648,484. Most preferably, at least two microneedles are used, and typically a linear array of at least three microneedles.

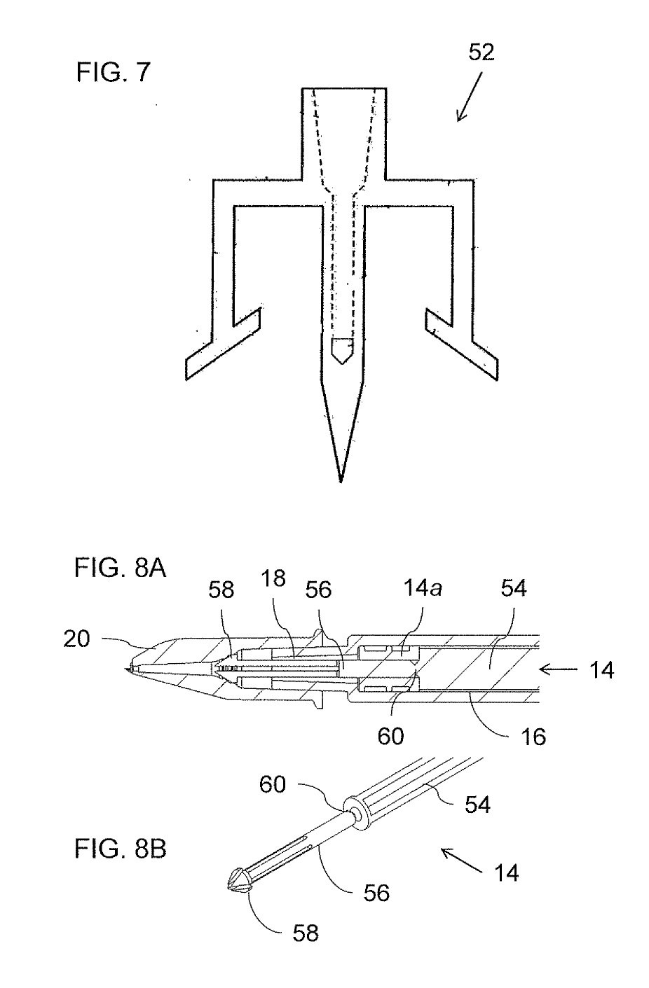

Filling of syringe 12 must be performed prior to attachment of microneedle adapter 20, typically by attachment of a dedicated filling adapter, which may be a filling needle or a vial adapter. The filling adapter must be releasably engaged with syringe 12 so that it can be disconnected after filling. By way of one non-limiting example, FIG. 7 illustrates a vial adapter 52 configured for releasable engagement with outlet 18 for filling of syringe 12. Vial adapter 52 as illustrated here is a slightly modified version of a vial adapter described in U.S. Pat. No. 5,279,576 where the length of the Luer connector has been shortened. This renders the vial adapter suitable for use with embodiments such as that of FIG. 6, so that the Luer connector stops short of ridge 48. Embodiments such as those of FIGS. 3-5 can also be used with vial adapter having an unmodified Luer connector. For the low-angle taper embodiment of FIG. 2, an alternative connector, for example with elastomeric O-ring seals, may be required to avoid locking together of the components. Other than the aforementioned minor adaptations to the Luer fitting, filling of syringe 12 can be performed with a range of otherwise conventional and commercially available vial adapters. Accordingly, the specific details of the vial adapter implementation are not part of the present invention, and will not be described here in detail.

In certain cases, microneedle drug delivery devices are valuable for delivering particularly small doses of drugs intradermally. The term “drug” is used herein in the broadest possible sense to include all compositions which are delivered into the body for therapeutic or other medically relevant effect. In such cases, and particularly for expensive drugs, reduction of dead space within the drug delivery device is of great importance. A range of possible dead-space-reducing inserts are disclosed in co-pending PCT Publication No. WO2010/067319, and may be used to advantage in the context of the present invention.

FIGS. 8A and 8B illustrate a further aspect of the present invention, useful in the context of the microneedle drug delivery devices of the present invention but not limited to such devices, in which a modified plunger structure provides a syringe with both dead-space reduction and auto-disable functionality.

FIG. 8A shows an assembly according to this aspect of the present invention with the distal portion of plunger 14 inserted within syringe 12 and an elastomeric seal 14a advanced to the end of barrel 16 at the end of the drug delivery stroke. FIG. 8B shows the distal portion of plunger 14 alone, with the elastomeric seal removed. Plunger 14 has a shaft 54 for driving seal 14a along the barrel so as to deliver a quantity of liquid through outlet 18, and a plunger extension 56 extending from the seal of the plunger and configured to advance within outlet 18 as the plunger is advanced. Plunger extension 56 terminates at a resilient tip 58 configured to extend beyond the outlet in a fully advanced position of the plunger. Resilient tip 58 is configured to expand laterally (i.e., perpendicular to the axis of outlet 18) so as to engage a region of the syringe around the outlet, thereby inhibiting withdrawal of the plunger extension. Shaft 54, plunger extension 56 and resilient tip 58 are preferably integrally formed as a single element, typically by an injection molding process. Resilience flexibility of the distal portion of plunger extension 56 is ensured by a number of slots, as shown. Most preferably, plunger 14 also features a reduced-strength region 60 located behind elastomeric seal 14a and configured to break when force is applied to withdraw the plunger after engagement of the resilient tip.

In use, the assembly is provided with the plunger in a forward position but just short of its locked state. Filling is performed using a suitable filling adapter by drawing the plunger back, the microneedle adapter (or in other applications, a regular needle) is connected to the outlet, and bubbles are purged from the syringe in the normal manner. The syringe is then ready for drug delivery.

As the plunger advances, plunger extension 56 advances within outlet channel 18 with its resilient tip 58 compressed, progressively contributing to reduction of the dead space within the drug delivery device. As the plunger reaches the end of its stroke, resilient tip 58 clears the end of outlet 18 and expands laterally/radially, thereby preventing withdrawal of the plunger. If significant force is applied in an attempt to draw back the plunger (e.g., for refilling), shaft 54 breaks away from plunger extension 56 at reduced-strength region 60, leaving the plunger seal 14a inaccessibly lodged at the end of the barrel and preventing re-use of the syringe.

Although described herein in the context of the implementation of FIGS. 8A and 8B, it should be noted that the present invention may be used to advantage with a wide range of otherwise conventional auto-disable syringes to provide additional protection against any attempt to refill the syringe.

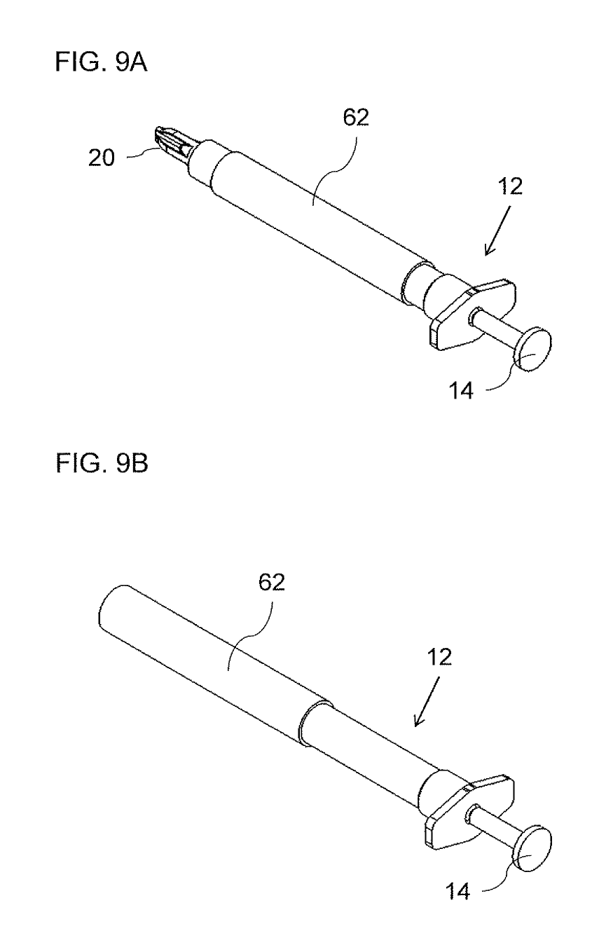

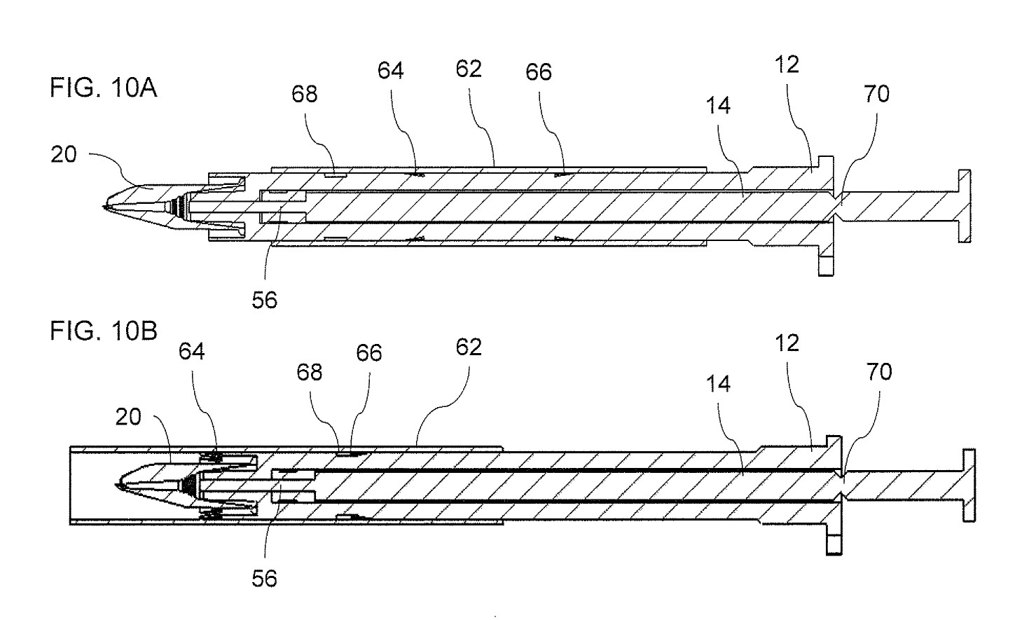

Turning now to FIGS. 9A-11B, there is shown a further optional but preferred feature for implementation in drug delivery devices according to the various aspects of the present invention. In this case, the drug delivery device further includes a sheath 62 circumscribing barrel 16. Sheath 62 is selectively displaceable from a normal position (FIGS. 9A and 10A) prior to and during drug delivery, to an advanced position (FIGS. 9B and 10B) in which sheath 62 covers microneedle adapter 20. Sheath 62 and syringe 12 having interlocking features configured to prevent retraction of the sheath from the advanced position.

The interlocking features are best seen in FIGS. 10A and 10B. Specifically, sheath 62 as shown here has a rearwardly-barbed front locking element 64 and a forwardly-barbed rear locking element 66. Barrel 14 has an annular recess 68. After use of the drug delivery device in the normal manner, sheath 62 is advanced manually to cover the microneedle adapter. As it is advanced, front locking element 64 passes over the end of the barrel 14 and locks against it to prevent withdrawal back to the normal position. The rear locking element 66 advances until it reaches and locks against annular recess 68. In this state, as illustrated in FIGS. 913 and 10B, sheath 62 is locked against significant further motion in either direction, and microneedle adapter 20 is hidden from view and rendered inaccessible. Optionally, a region of reduced strength 70 may be provided to allow snapping off the projecting portion of the plunger 14.

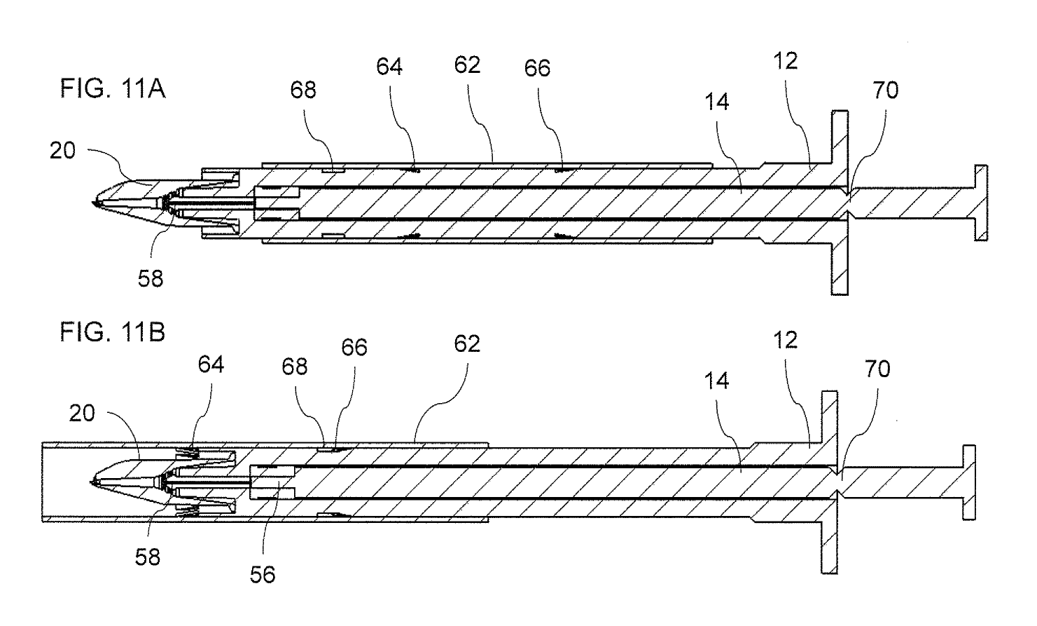

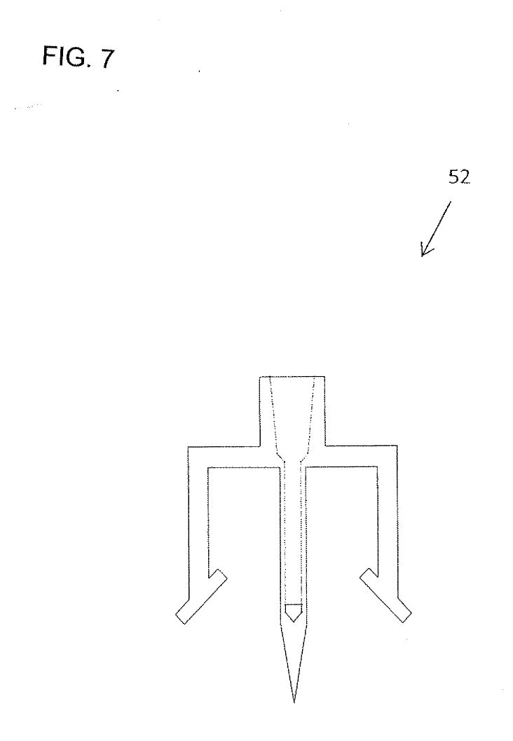

The embodiment of FIGS. 10A and 10B illustrates a plunger 14 with a plunger extension 56 for dead-space reduction only. However, it will be appreciated that this feature can be combined with the auto-disable option of FIGS. 8A and 8B. This combined implementation is illustrated in FIGS. 11A and 11B. Similarly, it should be noted that any of the features illustrated with reference to FIGS. 8A-11B can be combined to advantage with any of the irreversible interlocking configurations of FIGS. 2-6 to provide a particularly advantageous and synergous combination of auto-disable and/or safety features.

It should be noted that the various embodiments of the invention described above can be implemented using a wide range of materials. For example, possible syringe materials include but are not limited to glass and polymer (including PC, PP and others); possible hub materials include but are not limited to polymer (including PC, PP and others). Sealing elements are typically made from various elastomers, such as those commonly used in the industry. Silicone derivatives or rubbers could be employed for any such component. The drugs to be delivered may be anything that could be used in medicine, aesthetics and cosmetics. These could include liquid, and in some cases non-liquid, formulations or substances.

Additional elements such as safety syringe concepts, safety shields, safety needles, safety vial withdrawing systems and the like could be employed in combination with some of the embodiments.

The actuation of the different parts in some of the embodiments could be performed manually, but in various cases also mechanically (through spring or pressure mechanisms and others) and even electronically.

It will be appreciated that the above descriptions are intended only to serve as examples, and that many other embodiments are possible within the scope of the present invention as defined in the appended claims.

What is claimed is:

1. An intradermal drug delivery device comprising:
(a) a syringe having a plunger displaceable along a barrel for drawing a quantity of a liquid drug through an outlet and expelling the liquid drug through said outlet; and
(b) a microneedle adapter including at least one hollow microneedle, said microneedle adapter being configured to mate with said syringe so as to provide a leak-free flow path from said outlet through said at least one hollow microneedle for delivering the liquid drug intradermally,
wherein said microneedle adapter and said syringe are configured for irreversible engagement such that, after attachment of said microneedle adapter to said syringe, said microneedle adapter is resistant to non-destructive manual removal from said syringe.

2. The device of claim 1, wherein said outlet runs through a male conical fitting having a conical angle of less than 5.5%, and wherein said microneedle adapter is formed with a female conical fitting configured to mate with said male conical fitting.

3. The device of claim 1, wherein said outlet runs through a male conical fitting formed with a circumferential groove, and wherein said microneedle adapter is formed with a female conical fitting having at least one ridge, said female conical fitting being configured to mate with said male conical fitting with said at least one ridge engaging said groove.

4. The device of claim 1, wherein said outlet runs through a male conical fitting formed with at least one projecting ridge, and wherein said microneedle adapter is formed with a female conical fitting having a circumferential groove, said female conical fitting being configured to mate with said male conical fitting with said at least one ridge engaging said groove.

5. The device of claim 1, wherein said syringe is formed with at least one resilient engagement portion deployed to provide snap-engagement with a corresponding feature of said microneedle adapter.

6. The device of claim 1, wherein said microneedle adapter is formed with at least one resilient engagement portion deployed to provide snap-engagement with a corresponding feature of said syringe.

7. The device of claim 1, wherein said at least one hollow microneedle is integrally formed with a substrate from a single crystal material.

8. The device of claim 7, wherein said at least one hollow microneedle is formed with at least one upright surface, an inclined surface intersecting with said at least one upright surface, and a fluid flow bore extending through said substrate and intersecting with said inclined surface.

9. The device of claim 1, further comprising a vial adapter configured for releasable engagement with said outlet for filling of said syringe.

10. The device of claim 1, wherein said plunger is formed with a plunger extension extending from a seal of said plunger and configured to advance within said outlet as said plunger is advanced, thereby reducing a dead-space of the syringe.

11. The device of claim 10, wherein said plunger extension further comprises a resilient tip configured to extend beyond said outlet in a fully advanced position of said plunger, said resilient tip being configured to expand laterally so as to engage a region of said syringe around said outlet, thereby inhibiting withdrawal of said plunger extension.

12. The device of claim 11, wherein said plunger further comprises a reduced-strength region configured to break under traction applied to withdraw said plunger after engagement of said resilient tip.

13. The device of claim 1, wherein said syringe is an auto-disable syringe preventing refilling.

14. The device of claim 1, further comprising a sheath circumscribing said barrel, said sheath being selectively displaceable to an advanced position in which said sheath covers said microneedle adapter, said sheath and said syringe having interlocking features configured to prevent retraction of said sheath from said advanced position.

15. The device of claim 1, wherein said at least one hollow microneedle is implemented as a plurality of microneedles.

16. The device of claim 1, wherein said at least one hollow microneedle is implemented as a linear array of at least three microneedles.

17. An auto-disable syringe comprising:
(a) a syringe body comprising a barrel and terminating at an outlet; and
(b) a plunger having a shaft for driving a seal along said barrel so as to deliver a quantity of liquid through said outlet,
wherein said plunger is formed with a plunger extension extending from said seal of said plunger and configured to advance within said outlet as said plunger is advanced,
and wherein said plunger extension further comprises a resilient tip configured to extend beyond said outlet in a fully advanced position of said plunger, said resilient tip being configured to expand laterally so as to engage a region of said syringe around said outlet, thereby inhibiting withdrawal of said plunger extension.

18. The device of claim 17, wherein said shaft, said plunger extension and said resilient tip are integrally formed as a single element.

19. The device of claim 17, wherein said plunger further comprises a reduced-strength region configured to break when force is applied to withdraw said plunger after engagement of said resilient tip.
